(19) United States
(12) Patent Application Publication
(10) Pub. No.: US 2006/0154363 A1
(43) Pub. Date: Jul. 13, 2006

(54) Proliferation and delivery apparatus

(75) Inventor: Charles Henry Horn, Pretoria (ZA)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: AGRICULTURAL RESEARCH COUNCIL, PRETORIA (ZA)

(21) Appl. No.: 10/521,679

(22) PCT Filed: Jul. 15, 2003
(86) PCT No.: PCT/ZA03/00092

§ 371 Date: Sep. 12, 2005

(30) Foreign Application Priority Data
Jul. 18, 2002 (ZA) 02/5743

(51) Int. Cl. C12M 1/00; C12M 1/24

(52) U.S. Cl. 435/303.2; 435/304.2; 435/287.6

(57) ABSTRACT

A portable, disposable, unitary microorganism proliferation and delivery apparatus (10) is provided. The apparatus (10) comprises a proliferation chamber (12) for containing a growth medium (14); and an inoculation chamber (16) for containing an inoculum (18). The proliferation and inoculation chambers (12 and 16) are connected by a tube (19) defining a passage (20), which is closed by a separating means in the form of a septum (22). The inoculation chamber (16) is provided or defined by a flexible vial having a mouth (16.1) and the septum (22) closes the mouth. Opening means in the form of a spike (24) is mounted in the passage (20) directed at the septum (22). The mouth (16.1) of the inoculation chamber (16) is connected to one end of the tube (19) by an advancement means in the form of a screw thread connection (26).

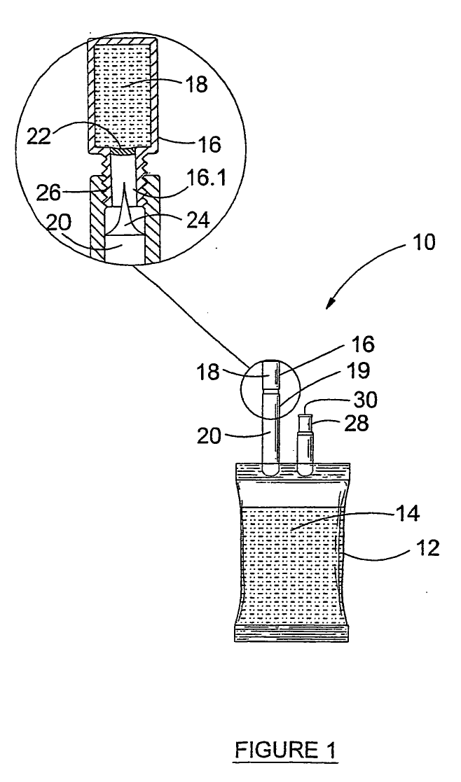

This invention relates to a unitary cell, tissue and/or microorganism proliferation and delivery apparatus and a method for proliferating and delivering cells, tissue cultures and/or microorganisms.

South Africa patent number 99/5408 discloses a bioreactor comprising a portable, disposable, transparent, extensile pouch having an inlet which is sealed by a seal. The construction of the bioreactor is preferably similar to or the same as that of an infusion bag. This invention may work under experimental conditions but is not viable for commercial proliferation and delivery of microorganisms at the point of use by novice end-users that do not have the necessary expertise or access to laboratory facilities and equipment, because of the need:
to introduce a suitable sterile medium;
to inoculate the medium with a suitable microorganism; and
for a separate delivery apparatus.

It would be extremely difficult for a novice commercial end-user to introduce the suitable sterile medium and do the inoculation without contaminating the bio-reactor with unwanted microorganisms or materials.

A further disadvantage of the known bioreactor is that it cannot exclude the risk of introducing the wrong medium or microorganism or the risk of contamination, since it is not a closed proliferation system. Furthermore, this invention does not mention that the bio-reactor per se can be used as a delivery apparatus. Also, the inoculation of the pouch with a microorganism strictly anaerobically is very difficult.

U.S. Pat. No. 6,245,555 number discloses a bioreactor for aseptic biological production or processing of cells, tissues and/or microorganisms. The bioreactor includes a support housing having an interior chamber; a disposable liner lining the interior chamber; and a head plate attached to the liner forming a sealed chamber with the liner. After use the liner can be disposed and the bioreactor can be reused with a new liner.

U.S. Pat. No. 6,391,638 number discloses a disposable device and method for anaerobically culturing and harvesting cells and/or tissue in consecutive cycles. The device consists of a sterilisable disposable container which may be partially filled with a suitable sterile biological cell and/or tissue culture medium. The container has means for removing excess air and/or waste gases therefrom, and means for introducing inoculum and/or culture medium and/or additives therein. A reusable harvesting means enables harvesting of at least a portion of the medium containing cells and/or tissue when desired, thereby enabling the device to be used continuously for at least one subsequent consecutive culturing/harvesting cycle. The portion of medium containing cells and/or tissue remaining from a previously harvested cycle may serve as inoculum for a next culture and harvest cycle.

Other types of bioreactors are disclosed in U.S. Pat. Nos. 5,763,267; 5,994,129; 6,228,635; 4,839,292; 5,416,022 and in EP 1022329.

A disadvantage of the above type of bioreactors is that although they may work under experimental conditions and in commercial laboratories, they are not commercially viable proliferation and delivery systems for use by novice end users because of the need:
to introduce a suitable sterile medium;
to inoculate the medium with a suitable microorganism; and
for a separate delivery apparatus.

In addition, these types of bioreactors:
are relatively complex and difficult to use and require microbiological expertise to operate;
present the risk of contamination with unwanted microorganisms or other materials;
present the risk of compromising the anaerobiosis of the bioreactors during the inoculation step;
present the risk of inoculation with an incorrect microorganism owing to an error or lack of knowledge, which could lead to substantial losses;
present the disadvantage that the inoculated culture has a limited shelf-life and has to be used within a limited time span; and
they are not portable and deliverable.

### OBJECT OF THE INVENTION

It is therefore an object of the present invention to provide a unitary cell, tissue and/or microorganism proliferation and delivery apparatus and a method for proliferating and delivering cells, tissue cultures and/or microorganisms with which the aforesaid disadvantages can be overcome or at least minimised.

### SUMMARY OF THE INVENTION

According to a first aspect of the invention there is provided a unitary cell, tissue and/or microorganism proliferation and delivery apparatus comprising:
at least one proliferation chamber for containing a growth medium;
at least one inoculation chamber for containing an inoculum; and
means for separating the proliferation and inoculation chambers, the separating means being openable to connect the insides of the chambers to each other to inoculate the growth medium with the inoculum, to allow proliferation of the cell, tissue and/or microorganism.

The applicants have found that the inoculum could be provided in a form which is stable and viable beyond the normal life-span of a conventional culture in a closed container, the arrangement being such that the inoculum and growth medium are stored and transported separated from each other in the apparatus, until such time as a proliferated culture is to be applied, whereupon the growth medium is inoculated and proliferation allowed to take place, whereafter the proliferated culture is dispensed from the apparatus.

The apparatus may be portable.

The apparatus may be disposable.

The growth medium may be cell, tissue and/or microorganism-specific.

The growth medium may be sterilised, pasteurised, filter sterilised, ultra high temperature sterilised, irradiated, and/or preserved prior to inoculation.

The separating means and inside of the proliferation chamber may also be sterile prior to inoculation.

Further according to the invention the chambers are anaerobic.

The apparatus may be provided with opening means for opening the separating means, without compromising the anaerobiosis of the inside of the chambers, the arrangement being such that the growth medium may be inoculated and the microorganism proliferated anaerobically and aseptically.

The chambers may be connected to each other via a passage.

The separating means may be in the form of a septum.

The opening means may be in the form of a spike for piercing the septum.

The inoculation chamber may be defined by a vial-type container having a mouth which is connected to one end of the passage.

The septum may cover the said mouth.

The vial-type container may be flexible, the arrangement being such that the inoculation chamber is compressed after the septum has been pierced to inoculate the growth medium. Alternatively, the apparatus may be provided with urging means for facilitating the inoculation step. Further alternatively a pressure drop between the two chambers may be utilised to facilitate the inoculation step.

The spike may be mounted in the passage directed at the septum, and the inoculation chamber may be connected to the said one end of the passage via advancement means, the arrangement being further such that, in use, the inoculation chamber is advanced inwardly towards the spike, until the spike pierces the septum.

The apparatus may be provided with a port for connecting to a dosing or application means.

The arrangement may further be such that pressure, which may build up in the proliferation chamber during the anaerobic cultivation of the microorganism, may urge the proliferated culture through the said port.

The proliferation chamber may be defined or provided by a flexible infusion bag type container. Alternatively the proliferation chamber may be in the form of a “carboy”-type container.

The apparatus may include additional proliferation inoculation chambers connectable to the other chambers.

The apparatus may be provided with an incubation means for controlling proliferation conditions of the inoculated growth media.

The inoculum may be a pure culture of a mixed culture.

The inoculum may be in the form of bacteria, viruses, fungi, or other microorganisms, or tissues, cells and the like.

According to a second aspect of the invention there is provided a method for the proliferation and delivery of cells, tissue cultures and/or microorganisms including the steps of:
disposing an inoculum in an inoculation chamber;
disposing a growth medium for the inoculum in a proliferation chamber which is separated from the inoculation chamber by an openable separating means;
storing and transporting the inoculum and uninoculated growth medium separated towards a point of use;
opening the separating means to inoculate the growth medium;
allowing the cells, tissue cultures and/or microorganisms to proliferate to form a proliferated culture; and
dispensing the proliferated culture from the proliferation chamber.

The method may include the further step of delivering the proliferated culture to a target locus.

The inoculation and proliferation chambers may be anaerobic and further according to the invention the steps of disposing, storing, transporting, inoculating, opening, and proliferation take place anaerobically.

The method may include the further step of controlling and/or adjusting proliferation conditions of the inoculated growth medium.

### BRIEF DESCRIPTION OF THE DRAWING

The invention will now be described further by way of a non-limiting example with reference to the accompanying drawing which is a side view of a unitary microorganism proliferation and delivery apparatus according to a preferred embodiment of the invention.

### DESCRIPTION OF A PREFERRED EMBODIMENT OF THE INVENTION

Referring to the drawing, a portable, disposable, unitary microorganism proliferation and delivery apparatus according to a preferred embodiment of the invention is generally designated by reference numeral 10.

The apparatus 10 comprises a proliferation chamber 12 for containing a growth medium 14; and an inoculation chamber 16 for containing an inoculum 18. The proliferation and inoculation chambers 12 and 16 are connected by a tube 19 defining a passage 20, which is closed by a separating means in the form of a septum 20. The inoculation chamber 16 is provided or defined by a flexible vial having a mouth 16.1 and the septum 22 closes the mouth. Opening means in the form of a spike 24 is mounted in the passage 20 directed at the septum 22. The mouth 16.1 of the inoculation chamber 16 is connected to one end of the tube 19 by an advancement means in the form of a screw thread connection 26.

The proliferation chamber 12 is provided or defined by a flexible infusion type bag and the apparatus 10 is further provided with a second tube 28 defining a port 30 for connecting to a dosing means (not shown) for administering the proliferated culture to a locus of use. The arrangement is further such that pressure which builds up in the proliferation chamber 12 during the cultivation of the microorganism, urges the proliferated culture through the said port 30. This obviates the need for external air pressure sources for dispensing the culture and thus decreases the risk of contamination.

The growth medium 14 is microorganism-specific and the inoculum 18 is in a stable form such as a freeze-dried culture, which is stable and viable beyond the normal life-span of a conventional culture in a closed container.

It will be appreciated that the proliferation chamber 12, growth medium 14, passage 22, and spike 24 are sterile prior to inoculation.

In use, the inoculation chamber 16 is screwed inwardly towards the spike 24, until the spike 24 pierces and opens the septum 22, to connect the insides of the chambers 12 and 16 to each other via the passage 20. Thereafter the inoculation chamber 16 is squeezed manually to inoculate the growth medium 14 with the inoculum 18, and proliferation of the microorganism is allowed. It will be appreciated that the spike 24 could be in the form of a hollow needle to provide an internal passage along which the inoculation can take place.

In the case of anaerobic microorganisms, and particularly strictly anaerobic microorganisms, the apparatus 10 is totally enclosed and hermetically sealed. Furthermore, the arrangement is such that the piercing of the septum 22, the inoculation of the growth medium 14, and the proliferation of the microorganism take place anaerobically.

Further in use, the inoculum 18 and uninoculated growth medium 14 are stored and transported separately from each other in the apparatus 10, until such time as a proliferated culture is to be applied, whereupon the growth medium 14 is inoculated as described above and proliferation allowed to take place, whereafter the proliferated culture is dispensed from the apparatus 10 via the port 30.

The applicants have found that the apparatus 10 presents several advantages over the conventional bioreactors. For example, the unitary and uncomplicated design of the apparatus allows for ease of use and it reduces the risk of contamination with unwanted microorganisms or other materials. It further reduces the risk of compromise of the anaerobic status of the chambers 12 and 16 during the inoculation step. It also ensures that the correct microorganisms at the correct levels are inoculated into the growth medium. In addition, it allows for an extended shelf life i.e. the inoculum and growth medium are stable until the septum is pierced.

In particular, because the inoculum and growth medium are stored and transported separated, and only inoculated shortly before delivery to the locus of use, a highly viable proliferated culture is provided. Because the microorganism in the proliferated culture is still in its log-growing phase, with its enzyme systems at an optimum, the effect thereof at the locus of delivery is substantially improved relative to the prior art systems.

It will be appreciated further that variations in detail are possible with a unitary cell, tissue and/or microorganism proliferation and delivery apparatus and a method for proliferating and delivering cells, tissue cultures and/or microorganisms according to the invention without departing from the scope of the appended claims. For example, the septum may by openable in any fashion, such as by dissolving it chemically, tearing, or bursting. Further for example, the separating means may be in the form of a valve.

1-32. (canceled)

33. An apparatus for the anaerobic proliferation and delivery of cells, tissue culture or microorganisms, the apparatus comprising an anaerobic proliferation chamber containing a growth medium, an inoculation chamber containing an inoculum, and an openable separator that separates the proliferation chamber from the inoculation chamber, wherein opening of the separator allows innoculation of the growth medium with the inoculum, wherein the inoculum is stable and viable beyond the normal life-span of a conventional culture in a closed container.

34. The apparatus of claim 33, wherein the apparatus is totally enclosed and hermetically sealed.

35. The apparatus of claim 33, wherein the separator and the inside of the proliferation chamber are sterile.

36. The apparatus of claim 33, wherein the inoculation chamber is anaerobic.

37. The apparatus of claim 33, further comprising an opener for opening the separator without compromising the anaerobiosis of the inside of the chambers.

38. The apparatus of claim 33, wherein the chambers are connected by a passage comprising the separator.

39. The apparatus of claim 37, wherein the separator comprises a septum.

40. The apparatus of claim 39, wherein the opener comprises a spike for piercing the septum.

41. The apparatus of claim 38, wherein the inoculation chamber comprises a vial-type container comprising a mouth connected to one end of the passage.

42. The apparatus of claim 41, wherein the septum covers the mouth.

43. The apparatus of claim 42, wherein a spike is mounted in the passage and directed at the septum, and wherein the mouth of the inoculation chamber is connected to the passage by an advancer that advances the inoculation chamber towards the spike thereby allowing the spike to pierce the septum.

44. The apparatus of claim 33, wherein the inoculation chamber is flexible and is compressible after the septum has been opened.

45. The apparatus of claim 44, further comprising an urging function that urges the inoculum into the proliferation chamber after the septum has been opened.

46. The apparatus of claim 33, wherein a pressure difference between the chambers causes the inoculum to flow into the proliferation chamber after the septum has been opened.

47. The apparatus of claim 46, wherein the pressure difference is created during the anaerobic cultivation of the cells, tissue culture or microorganisms in the inoculation chamber.

48. The apparatus of claim 33, further comprising a port for connecting to a dosing or application means.

49. The apparatus of claim 33 wherein the proliferation chamber comprises a flexible infusion bag-type container.

50. The apparatus of claim 33 wherein the proliferation chamber comprises a carboy-type container.

51. The apparatus of claim 33, further comprising an incubator for controlling the proliferation conditions of inoculated growth media.

52. A method for the proliferation and delivery of cells, tissue culture or microorganisms, the method comprising:
(a) disposing an inoculum in an inoculation chamber;
(b) disposing a growth medium for the inoculum in an anaerobic proliferation chamber which is separated from the inoculation chamber by an openable separator;
(c) opening the separator to inoculate the growth medium;
(d) allowing the cells, tissue cultures and/or microorganisms to proliferate under anaerobic conditions in the proliferation chamber to form a proliferated culture; and,
(e) dispensing the proliferated culture from the proliferation chamber.

53. The method of claim 52, wherein the inoculation chamber is anaerobic and wherein the steps of disposing, inoculating, opening, and proliferation occur anaerobically.

54. The method of claim 52, further comprising controlling proliferation conditions of the inoculated growth medium.

55. A unitary, disposable and portable apparatus for the proliferation and deliver of cells, tissue culture or microorganisms, the apparatus comprising an anaerobic proliferation chamber comprising a growth medium, an inoculation chamber comprising an inoculum, and a means for separating the proliferation and inoculation chambers, the separating means being openable to connect the insides of the chambers to each other to inoculate the growth medium with the inoculum, and to allow proliferation of the cells, tissue culture or microorganisms under anaerobic conditions, wherein the inoculum and growth medium are stored and transported separated from each other in the apparatus until such time as a proliferated culture is to be applied, whereupon the growth medium is inoculated and proliferation allowed to take place, whereafter the proliferated culture is dispensed from the apparatus.
